(19) United States
(12) Patent Application Publication
(10) Pub. No.: US 2026/0151135 A1
(43) Pub. Date: Jun. 4, 2026

(54) LEFT ATRIAL APPENDAGE CLOSURE DEVICES

(71) Applicant: BOSTON SCIENTIFIC SCIMED, INC., MAPLE GROVE, MN (US)

(72) Inventors: Troy Anthony Giese, Blaine, MN (US); Philip Andrew Litecky, Forest Lake, MN (US)

(73) Assignee: BOSTON SCIENTIFIC SCIMED, INC., MAPLE GROVE, MN (US)

(21) Appl. No.: 19/407,598

(22) Filed: Dec. 3, 2025

Related U.S. Application Data
(60) Provisional application No. 63/727,341, filed on Dec. 3, 2024.

(51) Int. Cl. A61B 17/12; A61B 17/00

(52) U.S. Cl. CPC A61B 17/1219; A61B 17/12031; A61B 17/12122; A61B 2017/00871

(57) ABSTRACT

A left atrial appendage closure device includes a shape memory foam element and a plurality of anchor elements. Each anchor element includes an elongate base portion and at least one tissue engaging portion. The elongate base portion is fixedly attached directly to the shape memory foam element. The plurality of anchor elements may be circumferentially spaced apart around the shape memory foam element. The elongate base portion may be at least partially disposed within a first slit formed in the shape memory foam element, or the elongate base portion may be disposed on and fixedly attached directly to an outer surface of the shape memory foam element.

### CROSS-REFERENCE TO RELATED APPLICATIONS

This application claims the benefit of priority of U.S. Provisional Application No. 63/727,341 filed December 3, 2024, the entire disclosure of which is hereby incorporated by reference.

### TECHNICAL FIELD

The disclosure relates generally to medical devices and more particularly to devices, systems, and methods that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices, systems, and methods, each has certain advantages and disadvantages. There is an ongoing need to provide alternative medical devices and systems, and methods for manufacturing and using medical devices and systems.

### SUMMARY

In one example, a left atrial appendage closure device may comprise a shape memory foam element configured for placement within a left atrial appendage, and a plurality of anchor elements.

Each anchor element of the plurality of anchor elements may comprise an elongate base portion and at least one tissue engaging portion extending from the elongate base portion. The elongate base portion may be at least partially disposed within a first slit formed in the shape memory foam element. The elongate base portion may be fixedly attached directly to the shape memory foam element.

In addition, or alternatively, to any example disclosed herein, the plurality of anchor elements is devoid of an interconnecting framework extending between elongate base portions of two or more anchor elements of the plurality of anchor elements.

In addition, or alternatively, to any example disclosed herein, the shape memory foam element has a tapered shape.

In addition, or alternatively, to any example disclosed herein, the shape memory foam element has a first outermost radial extent proximate a proximal end and a second outermost radial extent at a distal end that is less than the first outermost radial extent.

In addition, or alternatively, to any example disclosed herein, the at least one tissue engaging portion comprises a hook extending from the elongate base portion and having a tip extending toward a proximal end of the shape memory foam element.

In addition, or alternatively, to any example disclosed herein, at least a portion of the hook is recessed within a second slit formed in the shape memory foam element, wherein the second slit is oriented at an angle to the first slit.

In addition, or alternatively, to any example disclosed herein, the at least one tissue engaging portion comprises a plurality of teeth having tips extending toward a proximal end of the shape memory foam element.

In addition, or alternatively, to any example disclosed herein, the plurality of anchor elements is formed from a shape memory material.

In addition, or alternatively, to any example disclosed herein, the elongate base portion is fixedly attached to the shape memory foam element by adhesive bonding.

In addition, or alternatively, to any example disclosed herein, the elongate base portion is fixedly attached to the shape memory foam element by sutures.

In addition, or alternatively, to any example disclosed herein, the plurality of anchor elements extends radially outward from an outer surface of the shape memory foam element 0.762 mm (0.030 inches) or less.

In addition, or alternatively, to any example disclosed herein, and in a second example, a left atrial appendage closure device may comprise a shape memory foam element configured for placement within a left atrial appendage, and a plurality of anchor elements. Each anchor element of the plurality of anchor elements may comprise an elongate base portion and at least one tissue engaging portion extending from the elongate base portion. The elongate base portion may be at least partially disposed within a first slit formed in the shape memory foam element. The plurality of anchor elements may be circumferentially spaced apart around the shape memory foam element. The elongate base portion may be fixedly attached directly to the shape memory foam element.

In addition, or alternatively, to any example disclosed herein, the plurality of anchor elements is arranged in multiple rows spaced apart longitudinally along the shape memory foam element.

In addition, or alternatively, to any example disclosed herein, anchor elements within adjacent rows of the multiple rows are circumferentially offset from each other.

In addition, or alternatively, to any example disclosed herein, anchor elements within a selected row of the multiple rows are circumferentially spaced apart at equal angles from each other.

In addition, or alternatively, to any example disclosed herein, circumferential spacing between anchor elements varies from one row of the multiple rows to another row of the multiple rows.

In addition, or alternatively, to any example disclosed herein, the at least one tissue engaging portion is monolithically formed with the elongate base portion.

In addition, or alternatively, to any example disclosed herein, the plurality of anchor elements is arranged around the shape memory foam element in a pattern configured to prevent leaks around the shape memory foam element.

In addition, or alternatively, to any example disclosed herein, the plurality of anchor elements is devoid of an interconnecting framework configured to bias the plurality of anchor elements radially outward.

In addition, or alternatively, to any example disclosed herein, a left atrial appendage closure device may comprise a shape memory foam element configured for placement within a left atrial appendage, and a plurality of anchor elements. Each anchor element of the plurality of anchor elements may comprise an elongate base portion and at least one tissue engaging portion extending from the elongate base portion. The elongate base portion may be disposed on and fixedly attached directly to an outer surface of the shape memory foam element.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-4 schematically illustrate selected aspects of a left atrial appendage closure device;

FIG. 5 schematically illustrates selected aspects of an alternative configuration of the left atrial appendage closure device of FIGS. 1-4;

FIGS. 6-7 schematically illustrate selected aspects of a left atrial appendage closure device; and

FIG. 8 schematically illustrates selected aspects related to a left atrial appendage closure device disposed within a left atrial appendage.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the disclosure. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The devices, systems, and/or methods disclosed herein may provide a number of desirable features and benefits as described in more detail below.

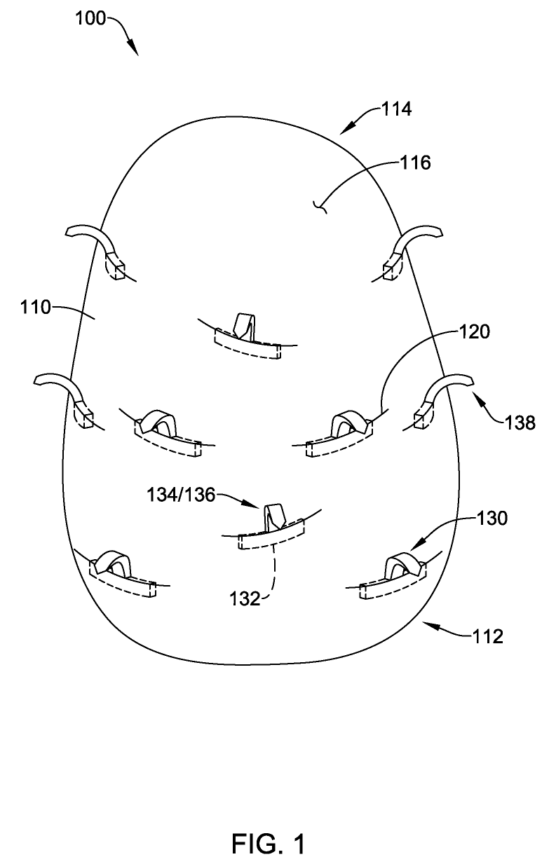

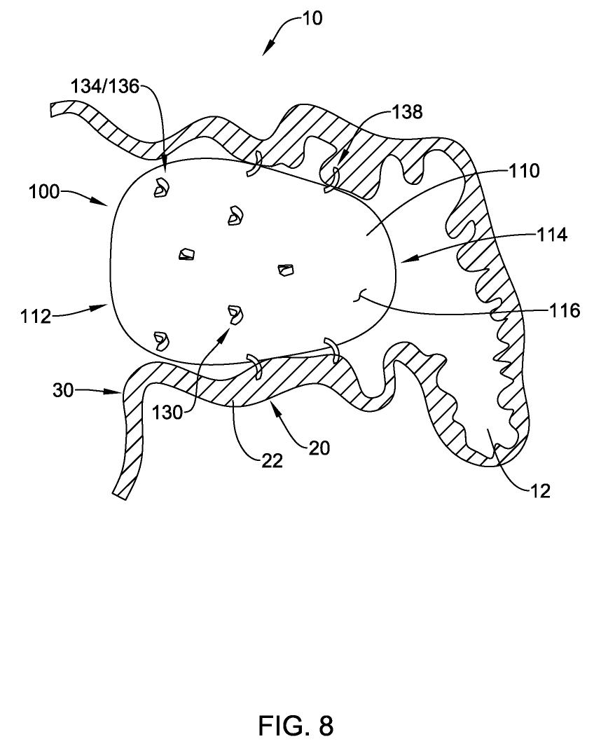

FIGS. 1-5 illustrate selected aspects of a left atrial appendage closure device 100. In some embodiments, the left atrial appendage closure device 100 may comprise a shape memory foam element 110 configured for placement within a left atrial appendage (e.g., FIG. 8). In some embodiments, the shape memory foam element 110 may extend from a first end 112 to a second end 114. In some embodiments, the first end 112 may be a proximal end of the left atrial appendage closure device 100 and/or the shape memory foam element 110, and the second end 114 may be a distal end of the left atrial appendage closure device 100 and/or the shape memory foam element 110.

In some embodiments, the shape memory foam element 110 may have a substantially homogenous construction. In some embodiments, the shape memory foam element 110 may comprise and/or may be formed from a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed or delivery size. In some examples, the collapsibility ratio of the shape memory polymer and/or the shape memory foam may be at least 5 times, at least 7 times, at least 8 times, at least 9 times, at least 10 times, at least 12 times, or more. Other configurations are also contemplated. In some embodiments, the shape memory foam element 110 may be configured as open celled foam.

In some embodiments, the shape memory foam element 110 and/or the shape memory foam may be formed from a biocompatible material. In some embodiments, the shape memory foam element 110 may be non-biodegradable and/or non-bioabsorbable. In some alternative embodiments, the shape memory foam element 110 may be biodegradable and/or bioabsorbable over time. In some embodiments, the shape memory foam element 110 may be configured to promote endothelization and/or tissue ingrowth. In some embodiments, the shape memory foam element 110 may include a coating, a material, and/or a component that promotes endothelization and/or tissue ingrowth. Other configurations are also contemplated.

In some embodiments, the shape memory foam element 110 may be configured to prevent thrombus formation. In some embodiments, the shape memory foam element 110 may include an anti-thrombus agent(s) and/or medicament(s). In some embodiments, the shape memory foam element 110 may be configured to absorb blood and/or bodily fluid(s). In some embodiments, the shape memory foam element 110 may be configured to trap thrombus. In some embodiments, the shape memory foam element 110 may be configured to promote tissue ingrowth and/or endothelization. Other configurations are also contemplated.

In some embodiments, the shape memory foam element 110 may optionally comprise a radiopaque marker coupled thereto and/or embedded therein. In some embodiments, the radiopaque marker may comprise a radiopaque substance or a radiopaque material disposed within the shape memory foam element 110 (e.g., the shape memory foam element 110 may be doped with and/or may include the radiopaque substance or the radiopaque material).

The left atrial appendage closure device 100 and/or the shape memory foam element 110 may be configured to shift from a collapsed configuration to an expanded configuration. The left atrial appendage closure device 100 and/or the shape memory foam element 110 may be disposed in the collapsed configuration during delivery. In some embodiments, the left atrial appendage closure device 100 and/or the shape memory foam element 110 may be constrained within a delivery device. In some embodiments, the left atrial appendage closure device 100 and/or the shape memory foam element 110 may be self-constrained in the collapsed configuration. Other configurations are also contemplated. The left atrial appendage closure device 100 and/or the shape memory foam element 110 may be configured to shift toward and/or to the expanded configuration after delivery and/or after being deployed within the left atrial appendage (e.g., in vivo). In some embodiments, the left atrial appendage closure device 100 and/or the shape memory foam element 110 may be configured to shift toward and/or to the expanded configuration when unconstrained. In some embodiments, the left atrial appendage closure device 100 and/or the shape memory foam element 110 may be configured to shift toward and/or to the expanded configuration upon exposure to a predetermined stimulus and/or changes therein (e.g., fluid, temperature, chemical environment, combinations thereof, etc.). Other configurations are also contemplated.

In some embodiments, the shape memory foam element 110 may be unconstrained by outside forces and/or structure(s) in the collapsed configuration. In some embodiments, the shape memory foam element 110 may be self-maintained in the collapsed configuration by shape memory properties. Other configurations are also contemplated.

In some embodiments, the shape memory foam element 110 may have a tapered shape in the expanded configuration and/or when unconstrained. In some embodiments, the shape memory foam element 110 may be tapered from the first end 112 toward and/or to the second end 114. In some embodiments, the shape memory foam element 110 may have a first outermost radial extent proximate the first end 112 and/or the proximal end of the shape memory foam element 110, and a second outermost radial extent at the second end 114 and/or the distal end of the shape memory foam element 110 that is less than the first outermost radial extent. In some embodiments, an outermost radial extent at a proximalmost end of the shape memory foam element 110 may be less than the first outermost radial extent (e.g., a proximal portion of the shape memory foam element 110 just distal of the proximalmost end may have a radial extent that is greater than at the proximalmost end of the shape memory foam element 110). In some embodiments, the shape memory foam element 110 may have a shape similar to and/or resembling a pine cone. Other configurations are also contemplated.

In some embodiments, the outer surface 116 of the shape memory foam element 110 may define a first overall volume in the collapsed configuration. In some embodiments, the outer surface 116 of the shape memory foam element 110 may define a second overall volume in the expanded configuration different from the first overall volume. In some embodiments, the second overall volume may be about 10% different, about 20% different, about 30% different, about 40% different, about 50% different, about 75% different, about 100% different, about 125% different, about 150% different, about 200% different, etc. from the first overall volume. In at least some embodiments, the second overall volume may be greater than the first overall volume.

In some embodiments, the shape memory foam element 110 may be substantially devoid of empty space or void space formed within the second overall volume defined by the outer surface 116 of the shape memory foam element 110 in the expanded configuration. In some embodiments, the second overall volume defined by the outer surface 116 of the shape memory foam element 110 in the expanded configuration may have less than 75% void space therein. In some embodiments, the second overall volume defined by the outer surface 116 of the shape memory foam element 110 in the expanded configuration may have less than 50% void space therein. In some embodiments, the second overall volume defined by the outer surface 116 of the shape memory foam element 110 in the expanded configuration may have less than 25% void space therein. In some embodiments, the second overall volume defined by the outer surface 116 of the shape memory foam element 110 in the expanded configuration may have less than 10% void space therein. In some embodiments, the second overall volume defined by the outer surface 116 of the shape memory foam element 110 in the expanded configuration may have less than 5% void space therein.

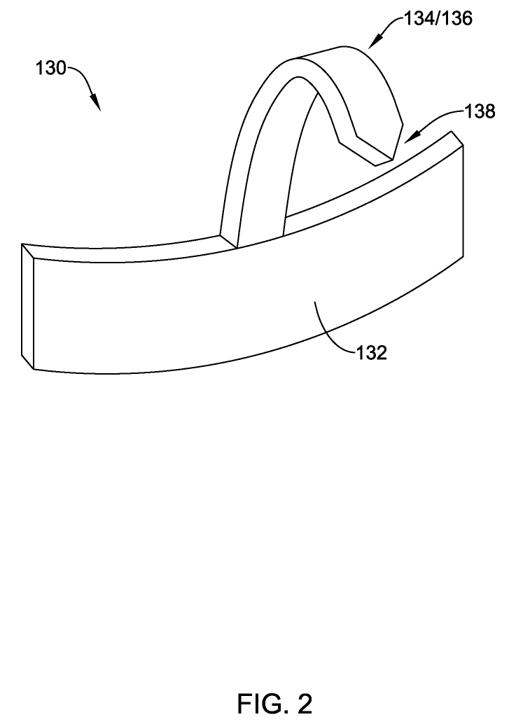

In some embodiments, the left atrial appendage closure device 100 may comprise a plurality of anchor elements 130, wherein each anchor element of the plurality of anchor elements 130 may comprise an elongate base portion 132 and at least one tissue engaging portion 134 extending from the elongate base portion 132, as seen in FIG. 2. In some embodiments, the at least one tissue engaging portion 134 may be monolithically formed with the elongate base portion 132.

In some embodiments, the elongate base portion 132 may be at least partially arcuate. In some alternative embodiments, the elongate base portion 132 may be substantially straight and/or may be substantially planar. Other configurations are also contemplated. In some embodiments, the at least one tissue engaging portion 134 may comprise a hook 136 extending from the elongate base portion 132 and may have a tip 138 extending toward the first end 112 and/or the proximal end of the shape memory foam element 110, as seen in FIG. 1 for example. In some alternative embodiments, the at least one tissue engaging portion 134 of one or more anchor elements of the plurality of anchor elements 130 may comprise a plurality of hooks extending from the elongate base portion 132. Other configurations are also contemplated.

Returning to FIG. 1, in some embodiments, the elongate base portion 132 may be at least partially disposed within a first slit 120 formed and/or cut in the shape memory foam element 110. In some embodiments, the elongate base portion 132 may be recessed within the first slit 120 formed in the shape memory foam element 110. In some embodiments, the elongate base portion 132 may be completely disposed within the first slit 120 formed in the shape memory foam element 110. In some embodiments, the shape memory foam element 110 may comprise a plurality of first slits (e.g., ref. 120) corresponding to and/or configured to receive the plurality of anchor elements 130 and/or the elongate base portions 132 thereof.

In at least some embodiments, the first slit 120 and/or the plurality of first slits may extend radially inward from the outer surface 116 of the shape memory foam element 110. In some embodiments, the first slit 120 and/or the plurality of first slits may extend at least partially in an axial direction and/or toward the first end 112 and/or the proximal end of the shape memory foam element 110 from the outer surface 116 of the shape memory foam element 110. In some embodiments, the first slit 120 and/or the plurality of first slits may extend into the shape memory foam element 110 generally parallel to a central longitudinal axis of the shape memory foam element 110 extending from the first end 112 and/or the proximal end of the shape memory foam element 110 to the second end 114 and/or the distal end of the shape memory foam element 110. In some embodiments, the first slit 120 and/or the plurality of first slits may be at least partially arcuate and/or may extend in an arc around the central longitudinal axis of the shape memory foam element 110 extending from the first end 112 and/or the proximal end of the shape memory foam element 110 to the second end 114 and/or the distal end of the shape memory foam element 110. Other configurations are also contemplated.

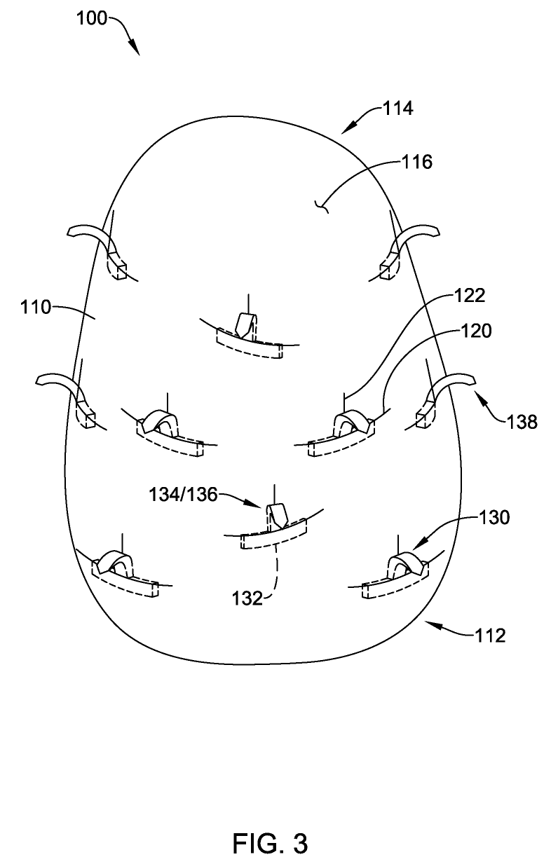

In some embodiments, at least a portion of the hook 136 may be recessed within a second slit 122 formed and/or cut in the shape memory foam element 110, as seen in FIG. 3. The second slit 122 may intersect the first slit 120. In some embodiments, the second slit 122 may be oriented at an angle to the first slit 120. In some embodiments, the second slit 122 may be oriented nonparallel to the first slit 120. In some embodiments, the second slit 122 may be oriented perpendicular to the first slit 120. In some alternative embodiments, the second slit 122 may be oriented at an oblique angle to the first slit 120. Other configurations are also contemplated. In some embodiments, the second slit 122 may be used and/or its depth into the shape memory foam element 110 from the outer surface 116 may be adjusted to change and/or control how far the plurality of anchor elements 130 and/or the at least one tissue engaging portion 134 extends radially outward from the outer surface 116 of the shape memory foam element 110.

In some embodiments, the plurality of anchor elements 130 and/or the at least one tissue engaging portion 134 may extend radially outward from the outer surface 116 of the shape memory foam element 110 about 0.762 millimeters (mm) (0.030 inches) or less. In some embodiments, the plurality of anchor elements 130 and/or the at least one tissue engaging portion 134 may extend radially outward from the outer surface 116 of the shape memory foam element 110 about 0.254 mm (0.010 inches). In some embodiments, the plurality of anchor elements 130 and/or the at least one tissue engaging portion 134 may extend radially outward from the outer surface 116 of the shape memory foam element 110 between about 0.254 mm (0.010 inches) and about 0.762 mm (0.030 inches). Other configurations are also contemplated. By limiting how far the plurality of anchor elements 130 and/or the at least one tissue engaging portion 134 extend radially outward from the outer surface 116 of the shape memory foam element 110, puncture of and/or penetration through the side wall of the left atrial appendage may be avoided. Additionally, compression and/or constraint of the left atrial appendage closure device 100 within a delivery device, as well as deployment of the left atrial appendage closure device 100 from the delivery device, may be easier and/or streamlined.

In some embodiments, the plurality of anchor elements 130 may be formed from a shape memory material. In some preferred embodiments, the plurality of anchor elements 130 may be formed from a metallic material. In some embodiments, the plurality of anchor elements 130 may be formed from a metallic shape memory material. In some alternative embodiments, the plurality of anchor elements 130 may be formed from a polymeric material or a composite material. Some suitable but non-limiting examples of materials for the plurality of anchor elements 130 are described below.

In some embodiments, the plurality of anchor elements 130 and/or the elongate base portion 132 may be fixedly attached directly to the shape memory foam element 110. In some embodiments, the plurality of anchor elements 130 and/or the elongate base portion 132 may be fixedly attached directly to the shape memory foam element 110 by adhesive bonding. In some embodiments, the plurality of anchor elements 130 and/or the elongate base portion 132 may be fixedly attached directly to the shape memory foam element 110 by one or more sutures. Other configurations are also contemplated.

In some embodiments, the plurality of anchor elements 130 may be circumferentially spaced apart around the shape memory foam element 110 and/or the central longitudinal axis of the shape memory foam element 110. In some embodiments, the plurality of anchor elements 130 may be arranged in multiple circumferential rows spaced apart longitudinally along the shape memory foam element 110. In some embodiments, the plurality of anchor elements 130 may be arranged in multiple circumferential rows axially spaced apart along the shape memory foam element 110. In some embodiments, anchor elements within adjacent circumferential rows of the multiple rows are circumferentially offset from each other. In some embodiments, anchor elements within adjacent circumferential rows of the multiple rows are circumferentially misaligned.

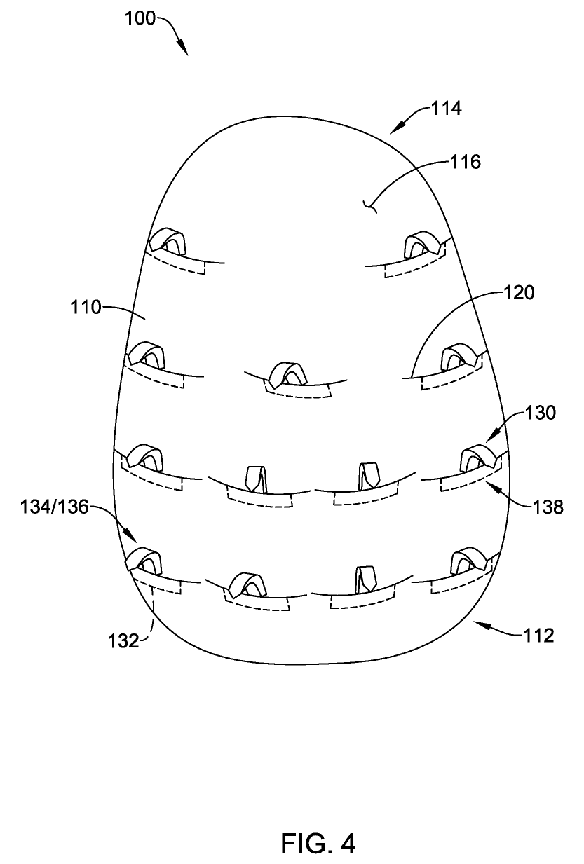

In some embodiments, two or more circumferential rows of the multiple circumferential rows may comprise an equal number of anchor elements. In some embodiments, two or more circumferential rows of the multiple circumferential rows may comprise a different number of anchor elements. In some embodiments, the density of the plurality of anchor elements 130 may increase toward the first end 112 and/or the proximal end of the shape memory foam element 110, as seen in FIG. 4. In some embodiments, anchor elements within a selected circumferential row of the multiple circumferential rows may be circumferentially spaced apart at equal angles from each other, the angles being measured between radii extending from the central longitudinal axis of the shape memory foam element 110 through a center of the anchor elements. In some embodiments, anchor elements within the selected circumferential row of the multiple circumferential rows may be spaced apart at 10-degree, 20-degree, 30-degree, 40-degree, 45-degree, 60-degree, 90-degree, 120-degree, or 180-degree intervals. In some embodiments, anchor elements within adjacent circumferential rows of the multiple circumferential rows may be circumferentially spaced apart at different angles from each other. In some embodiments, circumferential spacing between anchor elements may vary from one circumferential row of the multiple circumferential rows to another circumferential row of the multiple circumferential rows. Other configurations are also contemplated.

In some embodiments, the plurality of anchor elements 130 may be arranged around the shape memory foam element 110 in a pattern configured to prevent fluid leaks around the shape memory foam element 110. For example, as the plurality of anchor elements 130 engages with surrounding tissue, gaps or valleys could form between adjacent anchor elements of a selected circumferential row of anchor elements. Anchor elements in circumferential rows of anchor elements adjacent to the selected circumferential row may be positioned between anchor elements of the selected circumferential row, thereby closing and/or avoiding the gaps or valleys and preventing fluid leaks therethrough. Other configurations are also contemplated.

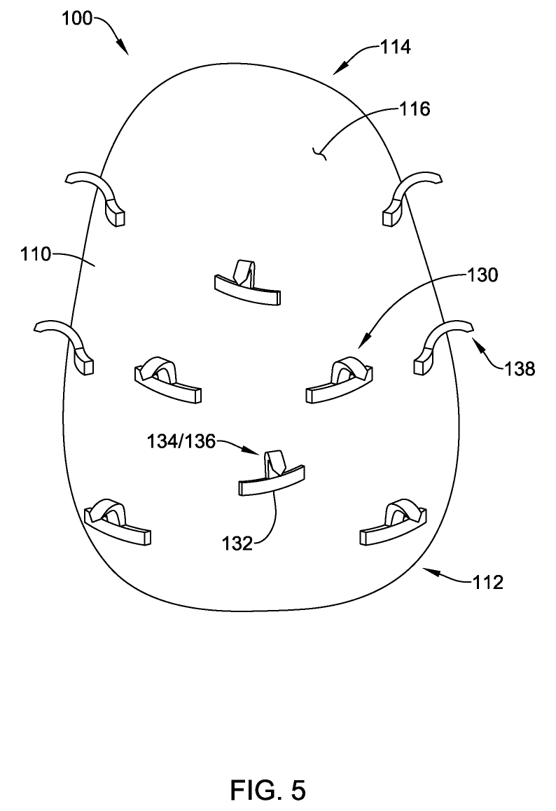

FIG. 5 illustrates an alternative configuration of the left atrial appendage closure device 100. In some embodiments, the plurality of anchor elements 130 and/or the elongate base portion 132 may be disposed on and fixedly attached directly to the outer surface 116 of the shape memory foam element 110. In some alternative embodiments, a first subset of the plurality of anchor elements 130 and/or the elongate base portion 132 may be disposed on and fixedly attached directly to the outer surface 116 of the shape memory foam element 110, and a second subset of the plurality of anchor elements 130 and/or the elongate base portion 132 may be at least partially disposed within the first slit 120 and/or the plurality of first slits, as described herein. Other configurations are also contemplated.

Referring back now to all of FIGS. 1-5, in some embodiments, the plurality of anchor elements 130 may be devoid of an interconnecting framework extending between elongate base portions (ref. 132) of two or more anchor elements of the plurality of anchor elements 130. In some embodiments, the elongate base portion 132 of each anchor element of the plurality of anchor elements 130 may not be coupled, connected, or attached to any other base portion via an interconnecting framework. For the purpose of this disclosure, the shape memory foam element 110 is expressly excluded from any definition or understanding of the term “interconnecting framework”. As such, base portions coupled, connected, or attached only to the shape memory foam element 110 shall not be considered to be coupled, connected, or attached to each other via an interconnecting framework. In some embodiments, the plurality of anchor elements 130 may be devoid of an interconnecting framework configured to bias the plurality of anchor elements 130 radially outward. In some embodiments, the plurality of anchor elements 130 may be devoid of an interconnecting framework configured to bias one or more of the plurality of anchor elements 130 radially outward. In some embodiments, the plurality of anchor elements 130 may be incapable of generating a radially outward force, except for that provided by the shape memory foam element 110 to which the plurality of anchor elements 130 is fixedly attached.

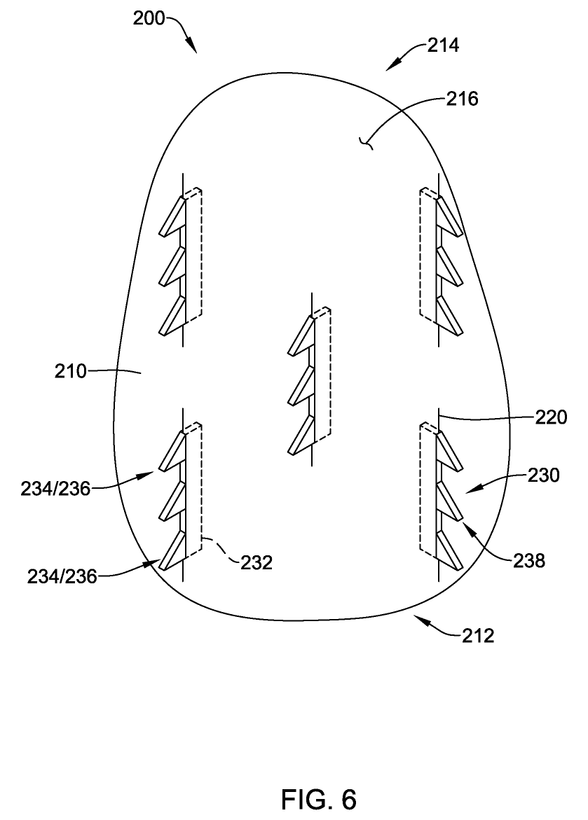

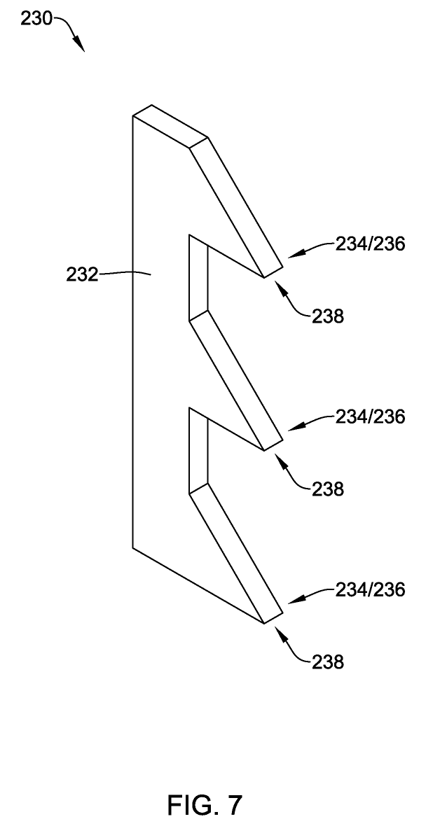

FIGS. 6-7 illustrate selected aspects of a left atrial appendage closure device 200. In some embodiments, the left atrial appendage closure device 200 may comprise a shape memory foam element 210 configured for placement within a left atrial appendage (e.g., FIG. 8). In some embodiments, the shape memory foam element 210 may extend from a first end 212 to a second end 214. In some embodiments, the first end 212 may be a proximal end of the left atrial appendage closure device 200 and/or the shape memory foam element 210, and the second end 214 may be a distal end of the left atrial appendage closure device 200 and/or the shape memory foam element 210.

In some embodiments, the shape memory foam element 210 may have a substantially homogenous construction. In some embodiments, the shape memory foam element 210 may comprise and/or may be formed from a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed or delivery size. In some examples, the collapsibility ratio of the shape memory polymer and/or the shape memory foam may be at least 5 times, at least 7 times, at least 8 times, at least 9 times, at least 10 times, at least 12 times, or more. Other configurations are also contemplated. In some embodiments, the shape memory foam element 210 may be configured as open celled foam.

In some embodiments, the shape memory foam element 210 and/or the shape memory foam may be formed from a biocompatible material. In some embodiments, the shape memory foam element 210 may be non-biodegradable and/or non-bioabsorbable. In some alternative embodiments, the shape memory foam element 210 may be biodegradable and/or bioabsorbable over time. In some embodiments, the shape memory foam element 210 may be configured to promote endothelization and/or tissue ingrowth. In some embodiments, the shape memory foam element 210 may include a coating, a material, and/or a component that promotes endothelization and/or tissue ingrowth. Other configurations are also contemplated.

In some embodiments, the shape memory foam element 210 may be configured to prevent thrombus formation. In some embodiments, the shape memory foam element 210 may include an anti-thrombus agent(s) and/or medicament(s). In some embodiments, the shape memory foam element 210 may be configured to absorb blood and/or bodily fluid(s). In some embodiments, the shape memory foam element 210 may be configured to trap thrombus. In some embodiments, the shape memory foam element 210 may be configured to promote tissue ingrowth and/or endothelization. Other configurations are also contemplated.

In some embodiments, the shape memory foam element 210 may optionally comprise a radiopaque marker coupled thereto and/or embedded therein. In some embodiments, the radiopaque marker may comprise a radiopaque substance or a radiopaque material disposed within the shape memory foam element 210 (e.g., the shape memory foam element 210 may be doped with and/or may include the radiopaque substance or the radiopaque material).

The left atrial appendage closure device 200 and/or the shape memory foam element 210 may be configured to shift from a collapsed configuration to an expanded configuration. The left atrial appendage closure device 200 and/or the shape memory foam element 210 may be disposed in the collapsed configuration during delivery. In some embodiments, the left atrial appendage closure device 200 and/or the shape memory foam element 210 may be constrained within a delivery device. In some embodiments, the left atrial appendage closure device 200 and/or the shape memory foam element 210 may be self-constrained in the collapsed configuration. Other configurations are also contemplated. The left atrial appendage closure device 200 and/or the shape memory foam element 210 may be configured to shift toward and/or to the expanded configuration after delivery and/or after being deployed within the left atrial appendage. In some embodiments, the left atrial appendage closure device 200 and/or the shape memory foam element 210 may be configured to shift toward and/or to the expanded configuration when unconstrained. In some embodiments, the left atrial appendage closure device 200 and/or the shape memory foam element 210 may be configured to shift toward and/or to the expanded configuration upon exposure to a predetermined stimulus and/or changes therein (e.g., fluid, temperature, chemical environment, combinations thereof, etc.). Other configurations are also contemplated.

In some embodiments, the shape memory foam element 210 may be unconstrained by outside forces and/or structure(s) in the collapsed configuration. In some embodiments, the shape memory foam element 210 may be self-maintained in the collapsed configuration by shape memory properties. Other configurations are also contemplated.

In some embodiments, the shape memory foam element 210 may have a tapered shape in the expanded configuration and/or when unconstrained. In some embodiments, the shape memory foam element 210 may be tapered from the first end 212 toward and/or to the second end 214. In some embodiments, the shape memory foam element 210 may have a first outermost radial extent proximate the first end 212 and/or the proximal end of the shape memory foam element 210, and a second outermost radial extent at the second end 214 and/or the distal end of the shape memory foam element 210 that is less than the first outermost radial extent. In some embodiments, an outermost radial extent at a proximalmost end of the shape memory foam element 210 may be less than the first outermost radial extent (e.g., a proximal portion of the shape memory foam element 210 just distal of the proximalmost end may have a radial extent that is greater than at the proximalmost end of the shape memory foam element 210). In some embodiments, the shape memory foam element 210 may have a shape similar to and/or resembling a pine cone. Other configurations are also contemplated.

In some embodiments, the outer surface 216 of the shape memory foam element 210 may define a first overall volume in the collapsed configuration. In some embodiments, the outer surface 216 of the shape memory foam element 210 may define a second overall volume in the expanded configuration different from the first overall volume. In some embodiments, the second overall volume may be about 10% different, about 20% different, about 30% different, about 40% different, about 50% different, about 75% different, about 100% different, about 125% different, about 150% different, about 200% different, etc. from the first overall volume. In at least some embodiments, the second overall volume may be greater than the first overall volume.

In some embodiments, the shape memory foam element 210 may be substantially devoid of empty space or void space formed within the second overall volume defined by the outer surface 216 of the shape memory foam element 210 in the expanded configuration. In some embodiments, the second overall volume defined by the outer surface 216 of the shape memory foam element 210 in the expanded configuration may have less than 75% void space therein. In some embodiments, the second overall volume defined by the outer surface 216 of the shape memory foam element 210 in the expanded configuration may have less than 50% void space therein. In some embodiments, the second overall volume defined by the outer surface 216 of the shape memory foam element 210 in the expanded configuration may have less than 25% void space therein. In some embodiments, the second overall volume defined by the outer surface 216 of the shape memory foam element 210 in the expanded configuration may have less than 10% void space therein. In some embodiments, the second overall volume defined by the outer surface 216 of the shape memory foam element 210 in the expanded configuration may have less than 5% void space therein.

In some embodiments, the left atrial appendage closure device 200 may comprise a plurality of anchor elements 230, wherein each anchor element of the plurality of anchor elements 230 may comprise an elongate base portion 232 and at least one tissue engaging portion 234 extending from the elongate base portion 232, as seen in FIG. 7. In some embodiments, the at least one tissue engaging portion 234 may be monolithically formed with the elongate base portion 232.

In some embodiments, the elongate base portion 232 may be substantially straight and/or may be substantially planar. In some embodiments, the elongate base portion 232 may be at least partially arcuate. Other configurations are also contemplated. In some embodiments, the at least one tissue engaging portion 234 may comprise a plurality of teeth 236 extending from the elongate base portion 232 and may have tips 238 extending toward the first end 212 and/or the proximal end of the shape memory foam element 210, as seen in FIG. 6 for example. In some alternative embodiments, the at least one tissue engaging portion 234 of one or more anchor elements of the plurality of anchor elements 230 may comprise a single tooth extending from the elongate base portion 232 instead of the plurality of teeth 236. Other configurations are also contemplated.

In some embodiments, the elongate base portion 232 may be at least partially disposed within a first slit 220 formed and/or cut in the shape memory foam element 210. In some embodiments, the elongate base portion 232 may be recessed within the first slit 220 formed in the shape memory foam element 210. In some embodiments, the elongate base portion 232 may be completely disposed within the first slit 220 formed in the shape memory foam element 210. In some embodiments, the shape memory foam element 210 may comprise a plurality of first slits (e.g., ref. 220) corresponding to and/or configured to receive the plurality of anchor elements 230 and/or the elongate base portions thereof.

In at least some embodiments, the first slit 220 and/or the plurality of first slits may extend radially inward from the outer surface 216 of the shape memory foam element 210. In some embodiments, the first slit 220 and/or the plurality of first slits may extend at least partially in a radially inward direction from the outer surface 216 of the shape memory foam element 210. In some embodiments, the first slit 220 and/or the plurality of first slits may extend into the shape memory foam element 210 generally perpendicular and/or normal to a central longitudinal axis of the shape memory foam element 210 extending from the first end 212 and/or the proximal end of the shape memory foam element 210 to the second end 214 and/or the distal end of the shape memory foam element 210. In some alternative embodiments, the first slit 220 and/or the plurality of first slits may be at least partially arcuate. Other configurations are also contemplated. In some embodiments, the first slit 220 and/or the plurality of first slits may be used and/or its depth into the shape memory foam element 210 from the outer surface 216 may be adjusted to change and/or control how far the plurality of anchor elements 230 and/or the at least one tissue engaging portion 234 extends radially outward from the outer surface 216 of the shape memory foam element 210.

In some embodiments, the plurality of anchor elements 230 and/or the at least one tissue engaging portion 234 may extend radially outward from the outer surface 216 of the shape memory foam element 210 about 0.762 mm (0.030 inches) or less. In some embodiments, the plurality of anchor elements 230 and/or the at least one tissue engaging portion 234 may extend radially outward from the outer surface 216 of the shape memory foam element 210 about 0.253 mm (0.010 inches). In some embodiments, the plurality of anchor elements 230 and/or the at least one tissue engaging portion 234 may extend radially outward from the outer surface 216 of the shape memory foam element 210 between about 0.254 mm (0.010 inches) and about 0.762 mm (0.030 inches). Other configurations are also contemplated. By limiting how far the plurality of anchor elements 230 and/or the at least one tissue engaging portion 234 extend radially outward from the outer surface 216 of the shape memory foam element 210, puncture of and/or penetration through the side wall of the left atrial appendage may be avoided. Additionally, compression and/or constraint of the left atrial appendage closure device 200 within a delivery device, as well as deployment of the left atrial appendage closure device 200 from the delivery device, may be easier and/or streamlined.

In some embodiments, the plurality of anchor elements 230 may be formed from a shape memory material. In some preferred embodiments, the plurality of anchor elements 230 may be formed from a metallic material. In some embodiments, the plurality of anchor elements 230 may be formed from a metallic shape memory material. In some alternative embodiments, the plurality of anchor elements 230 may be formed from a polymeric material or a composite material. Some suitable but non-limiting examples of materials for the plurality of anchor elements 230 are described below.

In some embodiments, the plurality of anchor elements 230 and/or the elongate base portion 232 may be fixedly attached directly to the shape memory foam element 210. In some embodiments, the plurality of anchor elements 230 and/or the elongate base portion 232 may be fixedly attached directly to the shape memory foam element 210 by adhesive bonding. In some embodiments, the plurality of anchor elements 230 and/or the elongate base portion 232 may be fixedly attached directly to the shape memory foam element 210 by one or more sutures. Other configurations are also contemplated.

In some embodiments, the plurality of anchor elements 230 may be circumferentially spaced apart around the shape memory foam element 210 and/or the central longitudinal axis of the shape memory foam element 210. In some embodiments, the plurality of anchor elements 230 may be arranged in multiple circumferential rows spaced apart longitudinally along the shape memory foam element 210. In some embodiments, the plurality of anchor elements 230 may be arranged in multiple circumferential rows axially spaced apart along the shape memory foam element 210. In some embodiments, the plurality of anchor elements 230 may be arranged in multiple circumferential rows that axially overlap along the shape memory foam element 210. In some embodiments, anchor elements within adjacent circumferential rows of the multiple rows are circumferentially offset from each other. In some embodiments, anchor elements within adjacent circumferential rows of the multiple rows are circumferentially misaligned.

In some embodiments, two or more circumferential rows of the multiple circumferential rows may comprise an equal number of anchor elements. In some embodiments, two or more circumferential rows of the multiple circumferential rows may comprise a different number of anchor elements. In some embodiments, the density of the plurality of anchor elements 230 may increase toward the first end 212 and/or the proximal end of the shape memory foam element 210, similar to the configuration shown in FIG. 4. In some embodiments, anchor elements within a selected circumferential row of the multiple circumferential rows may be circumferentially spaced apart at equal angles from each other, the angles being measured between radii extending from the central longitudinal axis of the shape memory foam element 210 through a center of the anchor elements. In some embodiments, anchor elements within the selected circumferential row of the multiple circumferential rows may be spaced apart at 10-degree, 20-degree, 30-degree, 40-degree, 45-degree, 60-degree, 90-degree, 120-degree, or 180-degree intervals. In some embodiments, anchor elements within adjacent circumferential rows of the multiple circumferential rows may be circumferentially spaced apart at different angles from each other. In some embodiments, circumferential spacing between anchor elements may vary from one circumferential row of the multiple circumferential rows to another circumferential row of the multiple circumferential rows. Other configurations are also contemplated.

In some embodiments, the plurality of anchor elements 230 may be arranged around the shape memory foam element 210 in a pattern configured to prevent fluid leaks around the shape memory foam element 210. For example, as the plurality of anchor elements 230 engages with surrounding tissue, gaps or valleys could form between adjacent anchor elements of a selected circumferential row of anchor elements. Anchor elements in circumferential rows of anchor elements adjacent to the selected circumferential row may be positioned between anchor elements of the selected circumferential row, thereby closing and/or avoiding the gaps or valleys and preventing fluid leaks therethrough. Other configurations are also contemplated.

In some embodiments, the plurality of anchor elements 230 may be devoid of an interconnecting framework extending between elongate base portions (ref. 232) of two or more anchor elements of the plurality of anchor elements 230. In some embodiments, the elongate base portion 232 of each anchor element of the plurality of anchor elements 230 may not be coupled, connected, or attached to any other base portion via an interconnecting framework. For the purpose of this disclosure, the shape memory foam element 210 is expressly excluded from any definition or understanding of the term “interconnecting framework”. As such, base portions coupled, connected, or attached only to the shape memory foam element 210 shall not be considered to be coupled, connected, or attached to each other via an interconnecting framework. In some embodiments, the plurality of anchor elements 230 may be devoid of an interconnecting framework configured to bias the plurality of anchor elements 230 radially outward. In some embodiments, the plurality of anchor elements 230 may be devoid of an interconnecting framework configured to bias one or more of the plurality of anchor elements 230 radially outward. In some embodiments, the plurality of anchor elements 230 may be incapable of generating a radially outward force, except for that provided by the shape memory foam element 210 to which the plurality of anchor elements 230 is fixedly attached.

FIG. 8 schematically illustrates one configuration of the left atrial appendage closure device 100 disposed within a left atrial appendage 10. It shall be understood that the left atrial appendage closure device 100 shown in FIG. 8 is merely exemplary, and any left atrial appendage closure device and/or embodiment or configuration thereof that is disclosed herein may be used in its place. Additionally, it shall be understood that the left atrial appendage 10 of FIG. 8 is merely exemplary, and other geometries, shapes, sizes, etc. for the left atrial appendage 10 are common and may vary from patient to patient. The left atrial appendage 10 may be formed as a small pouch or extension attached to and extending from the left atrium of a patient’s heart.

The left atrial appendage 10 may include a longitudinal axis arranged along a depth of a main body 20 of the left atrial appendage 10. The main body 20 may include a side wall 22 and an ostium 30 forming a proximal mouth. In some embodiments, a lateral extent of the main body 20 and/or the ostium 30 may be smaller or less than a depth of the main body 20 along the longitudinal axis, or a depth of the main body 20 may be greater than a lateral extent of the main body 20 and/or the ostium 30. In some embodiments, the left atrial appendage 10 may narrow quickly along the depth of the main body 20 or the left atrial appendage 10 may maintain a generally constant lateral extent along a majority of the depth of the main body 20.

In some embodiments, the left atrial appendage 10 may include a distalmost region 12 formed or arranged as a tail-like element associated with a distal portion of the main body 20. In some embodiments, the distalmost region 12 may protrude radially or laterally away from the longitudinal axis and/or the main body 20.

As seen in FIG. 8, the left atrial appendage closure device 100 may be deployed within the left atrial appendage 10 proximate the ostium 30. In some embodiments, the shape memory foam element 110 may be configured to adapt and conform to the left atrial appendage 10 and/or surrounding anatomy when shifting from the collapsed configuration toward and/or to the expanded configuration in vivo. In some embodiments, the tip 138 of the at least one tissue engaging portion 134 of the left atrial appendage closure device 100 may be configured to engage and/or penetrate the side wall 22 in multiple locations within the left atrial appendage to secure the left atrial appendage closure device 100 within the left atrial appendage 10.

In some embodiments, the left atrial appendage closure device 100 and/or the shape memory foam element 110 may be disposed within the ostium 30 and/or may fill and/or block the proximal mouth of the ostium 30. In some embodiments, the left atrial appendage closure device 100 and/or the shape memory foam element 110 may define a proximalmost surface of the left atrial appendage closure device 100 and/or the shape memory foam element 110 and/or may be configured to face outward from the left atrial appendage 10 and/or to face toward the left atrium. In some embodiments, the proximalmost surface of the left atrial appendage closure device 100 and/or the shape memory foam element 110 may comprise and/or may be coated with a therapeutic agent. In some embodiments, other surfaces of the left atrial appendage closure device 100 and/or the shape memory foam element 110 (e.g., surfaces other than the proximalmost surface) may comprise and/or may be coated with a therapeutic agent. In some embodiments, the proximalmost surface of the left atrial appendage closure device 100 and/or the shape memory foam element 110 and the other surfaces of the left atrial appendage closure device 100 and/or the shape memory foam element 110 may comprise and/or may be coated with different therapeutic agents. Other configurations are also contemplated.

The materials that can be used for the various components of the system (and/or other elements disclosed herein) and the various components thereof disclosed herein may include those commonly associated with medical devices, implants, and/or systems. For simplicity purposes, the following discussion refers to the system. However, this is not intended to limit the devices and methods described herein, as the discussion may be applied to other elements, members, components, or devices disclosed herein, such as, but not limited to, the left atrial appendage closure device, the shape memory foam element, the plurality of anchor elements, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

The shape memory foam element may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. Suitable transition temperatures may be, for example, at or below about 37 °C (about 98.6 °F), which allows the shape memory foam to assume an initial configuration prior to and during delivery through a delivery catheter or other delivery device, and an expanded configuration for occlusion after delivery and release within the left atrial appendage, allowing the foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the foam in the expanded configuration will be from about 10 kg/m3 (about 0.62 lb/ft3) to about 1000 kg/m3 (about 62.31 lb/ft3), including from about 10 kg/m3 to about 500 kg/m3 (about 31.2 lb/ft3) including from about 10 kg/m3 to about 200 kg/m3 (about 12.5 lb/ft3), including from about 20 kg/m3 to about 100 kg/m3 (about 6.2 lb/ft3).

Generally, the material for constructing the shape memory foam element is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam element may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam element may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerization of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha,omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam element may include thermoset polyurethanes that include oxidatively susceptible linkages in the soft segment, including but not limited to tertiary amines and polyethers. The shape memory foam element may optionally include hydrolytically degradable soft segment components such as polycaprolactone, esters, and others. In some cases, the polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers, and supramolecular networks, among others. In some instances, shape memory polymers that may be used in creating the foamable solutions described herein may include polyurethane, for example.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. A left atrial appendage closure device, comprising:
a shape memory foam element configured for placement within a left atrial appendage; and
a plurality of anchor elements, wherein each anchor element of the plurality of anchor elements comprises an elongate base portion and at least one tissue engaging portion extending from the elongate base portion;
wherein the elongate base portion is at least partially disposed within a first slit formed in the shape memory foam element;
wherein the elongate base portion is fixedly attached directly to the shape memory foam element.

2. The left atrial appendage closure device of claim 1, wherein the plurality of anchor elements is devoid of an interconnecting framework extending between elongate base portions of two or more anchor elements of the plurality of anchor elements.

3. The left atrial appendage closure device of claim 1, wherein the shape memory foam element has a tapered shape.

4. The left atrial appendage closure device of claim 1, wherein the shape memory foam element has a first outermost radial extent proximate a proximal end and a second outermost radial extent at a distal end that is less than the first outermost radial extent.

5. The left atrial appendage closure device of claim 1, wherein the at least one tissue engaging portion comprises a hook extending from the elongate base portion and having a tip extending toward a proximal end of the shape memory foam element.

6. The left atrial appendage closure device of claim 5, wherein at least a portion of the hook is recessed within a second slit formed in the shape memory foam element, wherein the second slit is oriented at an angle to the first slit.

7. The left atrial appendage closure device of claim 1, wherein the at least one tissue engaging portion comprises a plurality of teeth having tips extending toward a proximal end of the shape memory foam element.

8. The left atrial appendage closure device of claim 1, wherein the plurality of anchor elements is formed from a shape memory material.

9. The left atrial appendage closure device of claim 1, wherein the elongate base portion is fixedly attached to the shape memory foam element by adhesive bonding.

10. The left atrial appendage closure device of claim 1, wherein the elongate base portion is fixedly attached to the shape memory foam element by sutures.

11. The left atrial appendage closure device of claim 1, wherein the plurality of anchor elements extends radially outward from an outer surface of the shape memory foam element 0.762 mm (0.030 inches) or less.

12. A left atrial appendage closure device, comprising:
a shape memory foam element configured for placement within a left atrial appendage; and
a plurality of anchor elements, wherein each anchor element of the plurality of anchor elements comprises an elongate base portion and at least one tissue engaging portion extending from the elongate base portion;
wherein the elongate base portion is at least partially disposed within a first slit formed in the shape memory foam element;
wherein the plurality of anchor elements is circumferentially spaced apart around the shape memory foam element;
wherein the elongate base portion is fixedly attached directly to the shape memory foam element.

13. The left atrial appendage closure device of claim 12, wherein the plurality of anchor elements is arranged in multiple rows spaced apart longitudinally along the shape memory foam element.

14. The left atrial appendage closure device of claim 13, wherein anchor elements within adjacent rows of the multiple rows are circumferentially offset from each other.

15. The left atrial appendage closure device of claim 13, wherein anchor elements within a selected row of the multiple rows are circumferentially spaced apart at equal angles from each other.

16. The left atrial appendage closure device of claim 15, wherein circumferential spacing between anchor elements varies from one row of the multiple rows to another row of the multiple rows.

17. The left atrial appendage closure device of claim 12, wherein the at least one tissue engaging portion is monolithically formed with the elongate base portion.

18. The left atrial appendage closure device of claim 12, wherein the plurality of anchor elements is arranged around the shape memory foam element in a pattern configured to prevent leaks around the shape memory foam element.

19. The left atrial appendage closure device of claim 1, wherein the plurality of anchor elements is devoid of an interconnecting framework configured to bias the plurality of anchor elements radially outward.

20. A left atrial appendage closure device, comprising:
a shape memory foam element configured for placement within a left atrial appendage; and
a plurality of anchor elements, each anchor element of the plurality of anchor elements comprises an elongate base portion and at least one tissue engaging portion extending from the elongate base portion;
wherein the elongate base portion is disposed on and fixedly attached directly to an outer surface of the shape memory foam element.
